(19) United States
(12) Patent Application Publication
(10) Pub. No.: US 2010/0143981 A1
(43) Pub. Date: Jun. 10, 2010

(54) WHOLE-CELL CATALYTIC SYSTEM COMPRISING A HYDANTOINASE, A RACEMASE AND A CARBAMOYLASE

(76) Inventors: Monika Rusnak-Müller, Aachen (DE); Oliver May, Aachen (DE); Petrus Johannes Hermsen, Horst (NL); Henricus Martinus Maria Gerardus Straatman, Horst (NL); Wolfgang Skranc, Vienna (AT); Wilhelmus Hubertus Joseph Boesten, Sittard (NL); Dannis Heemskerk, Schinveld (NL); Ben De Lange, Munstergeleen (NL); Georgios Sarakinos, Munich (DE)

Correspondence Address: NIXON & VANDERHYE, PC, 901 NORTH GLEBE ROAD, 11TH FLOOR, ARLINGTON, VA, 22203, US

(21) Appl. No.: 12/517,504

(22) PCT Filed: Dec. 4, 2007
(86) PCT No.: PCT/EP2007/010511

§ 371 Date: Dec. 11, 2009

(30) Foreign Application Priority Data
Dec. 4, 2006 (EP) 06025019.8
Jun. 6, 2007 (EP) 07109685.3

(51) Int. Cl. C12P 13/04; C12N 1/21; C12N 15/74; C07D 233/72; C07C 205/01

(52) U.S. Cl. 435/106; 435/252.33; 435/320.1; 548/317.1; 548/319.5; 562/553

(57) ABSTRACT

The present invention relates to a whole cell catalytic system for the preparation of an enantiomerically enriched α-amino acid from a corresponding hydantoin wherein hydantoinase, L-carbamoylase and hydantoin racemase are coexpressed in a recombinant micro-organism wherein the genes coding for these three enzymes are located on a single replicon. The present invention further relates to the use of such a whole cell catalytic system according in the preparation of an enantiomerically enriched L-α-amino acid from a corresponding hydantoin.

The present invention relates to a whole cell catalytic system for the preparation of an enantiomerically enriched α-amino acid from a corresponding hydantoin and to the use of such whole cell catalytic system for said conversion.

Whole cell catalytic systems for the preparation of an enantiomerically enriched α-amino acid from a corresponding hydantoin are known from WO01/23582. According to WO01/23582 E. coli has been equipped with genetic material encoding the three enzymes hydantoinase, carbamoylase and hydantoin racemase of Arthrobacter aurescens DSM 3747 and the genes are overexpressed in the cells according to their turnover rates. This was done in order to “fine tune” the expression of the genes. More in particular this publication discloses the expression of the three genes from the same promoter but from separate replicons with different copy numbers.

The present invention relates to an improvement of such whole cell catalytic system for the preparation of an enantiomerically enriched L-α-amino acid from a corresponding hydantoin. According to the present invention hydantoinase, L-carbamoylase and hydantoin racemase are coexpressed in a recombinant micro-organism wherein the genes coding for these three enzymes are located on a single replicon.

According to a further embodiment of the present invention the hydantoin racemase is derived from a gene of an Agrobacterium species. In a preferred amodiment, the hydantoin racemase is derived from a gene of an Agrobacterium species and the hydantoinase and L-carbamoylase are each independently derived from genes from species other than an Agrobacterium species. Suitbale species to derive genes for the hydantoinase and L-carbamoylase from are for example Arthrobacter, Pseudemonas and Bacillus.

Such a whole cell catalytic system is particularly useful for the preparation of an enantiomerically enriched L-α-amino acid starting from a substrate with the general formula [1]

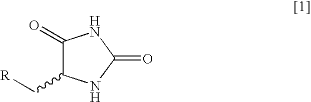

wherein R denotes a substituent with at least 3 carbon atoms, optionally containing further one or more hetero atoms, one or more double bonds and/or one or more cyclic structures. It is an advantage of the method according to the invention, that both enantiomers of the compound according to formula [1] can be converted in the desired S-amino acid.

This particular combination of genes on a single replicon has never been applied to the preparation of an enantiomerically enriched L-α-amino acid from a corresponding hydantoin, and it was surprising that such conversion was possible at a high yield even without fine tuning of the copy numbers as described to be necessary according to the prior art cited above.

Suitably the three genes encoding the respective enzymes hydantoinase, L-carbamoylase and hydantoin racemase are under the control of a single promoter. Promoters useful according to the present invention are promoters suited for expression of genes in the particular recombinant micro-organism. Examples of such promoters are inducible promoters for operons/genes like the lactose operon (lac), the rhamnose operon (rha), the arabinose operon (ara), the tryptophan operon (trp), the operon encoding enzymes common to the biosynthesis of all aromatic amino acids (aro), or functional hybrids of these. Other examples of useful promoters are constitutive promoters.

According to a further embodiment of the present invention the single replicon contains SEQ ID NO: 2 encoding L-hydantoinase, SEQ ID NO: 3 encoding L-carbamoylase and SEQ ID NO: 4 encoding hydantoin racemase.

It will be obvious to a person skilled in the art that whenever in this text sequences are identified, the invention also relates to all sequences with a homology of 90%, more preferably 95%, 96%, 97%, 98% or 99% to the identified sequence. For the purpose of this invention, homology is determined according to the procedure described by Tatiana A. Tatusova and Thomas L. Madden “Blast 2 sequences—a new tool for comparing protein and nucleotide sequences”, FEMS Microbiol Lett. 174:247-250 (1999), The following are the standard parameters used at http://www.ncbi.nlm.nih.gov/BLAST/bl2seq/wblast2.cgifor Protein sequences:
Matrix: BLOSUM62
Open gap: 5
extension gap: 2
Penalties gap x_dropoff: 11
Expected: 10
word size: 11

DNA Sequences with a homology of 90%, more preferably 95% 96%, 97%, 98% or 99% to the DNA sequences according to the invention are explicitly also part of the invention for the DNA constructs according to the invention. The following standard parameters are thereby used at http://www.ncbi.nlm.nih.gov/BLAST/bl2seq/wblast2.cgifor Protein sequences:
Reward for match: 1
Penalty for mismatch: −2
Open gap: 11
Extension gap: 1
Penalties gap x_dropoff: 50
Expected: 10
Word size: 3

The present invention also relates to a DNA construct containing the sequences encoding hydantoinase, L-carbamoylase and hydantoin racemase operationally linked to a single promoter. More in particular the invention relates to a DNA construct wherein the sequence encoding hydantoin racemase is derived from an Agrobacterium species. More specifically the DNA construct may contain the sequences encoding hydantoinase of the sequence represented by SEQ ID NO: 2, carbamoylase of the sequence represented by SEQ ID NO: 3 and racemase of the sequence represented by SEQ ID NO: 4.

In a further embodiment the present invention relates to an expression vector containing a DNA construct described above.

The DNA sequences encoding the respective enzymes may be the sequences occurring naturally, or may be synthetic sequences. The synthetic sequences may be adapted compared to the naturally occurring sequences e.g. by using codons for the amino acids which are more suitable for expression in the particular recombinant microorganism selected for the whole cell catalytic systems of the present invention.

The replicon for use according to the present invention can be a plasmid, a phage or a chromosome. The replicon may comprise a marker enabling selection of cells harboring the element, as well as DNA sequences responsible for autonomous propagation and or equal distribution of the element within the host cell and its daughter cells.

Suitable microorganisms for use according to the invention are prokaryotes and in particular bacteria, and more in particular E. coli.

The present invention also relates to the use of a whole cell catalytic system as described above in the preparation of enantiomerically enriched α-amino acids from a corresponding hydantoin.

In particular, the invention relates to the use of said whole cell catalytic system for the preparation of an enantiomerically enriched L-α-amino acid of general formula [2]

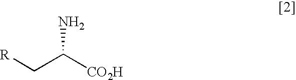

starting from a substrate with the general formula [1]

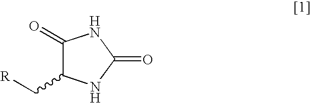

wherein R has the meaning given above.

More in particular the invention also relates to the use of the whole cell catalytic system in the conversion of a hydantoin of formula [3]

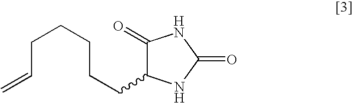

into an L-α-amino acid of formula [4]

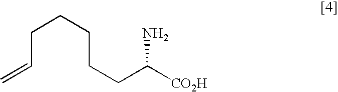

wherein R has the meaning given above.

The present invention also concerns compounds of formula [3] and [4].

Also, the invention relates to the use of the whole cell catalytic system in the conversion of a hydantoin of formula [5a] or [5b]

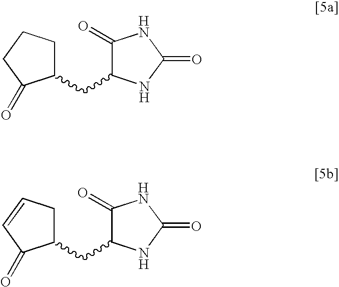

into an L-α-amino acid of formula [6a] or [6b] respectively

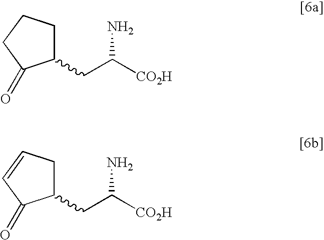

L-α-amino acids of formula [6a] and [6b] can be used as intermediates in the synthesis of the angiotensin converting enzyme (ACE) inhibitor ramipril. In most prior art methods ramipril is prepared starting from racemic [6a], requiring a subsequent optical resolution that inevitably results in loss of approximately 50% of the end-product, a problem that is solved by using L-α-amino acid of formula [6a] or [6b]. Thus, in one embodiment a method is presented for the conversion of L-α-amino acid of formula [6a] or [6b] by hydrogenation into (2S,3aS,6aS)-octahydrocyclopenta[b]pyrrole-2-carboxylic acid, which is a building block common in all commercially viable routes towards ramipril known today. In a preferred embodiment, hydrogenation of L-α-amino acid of formula [6a] or [6b] is carried out using hydrogen gas in the presence of a suitable catalyst such as for example palladium on charcoal.

Furthermore, the invention relates to the use of the whole cell catalytic system in the conversion of a hydantoin of formula [7a] or [7b]

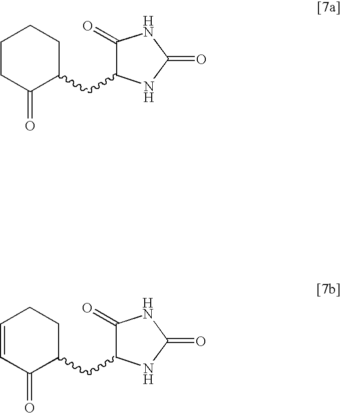

into an L-α-amino acid of formula [8a] or [8b] respectively

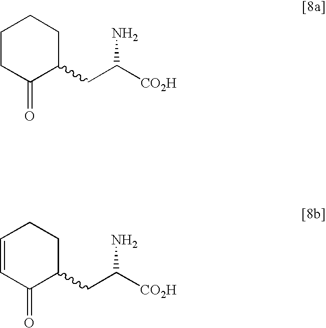

L-α-amino acids of formula [8a] and [8b] can be used as intermediates in the synthesis of the angiotensin converting enzyme (ACE) inhibitor perindopril, with similar advantages as described above for L-α-amino acids of formula [6]. Thus, in one embodiment a method is presented for the conversion of L-α-amino acid of formula [8a] or [8b] by hydrogenation into (2S,3aS,6aS)-decahydrocyclohexa[b]pyrrole-2-carboxylic acid, which is a building block common in all commercially viable routes towards perindopril known today.

Also, the invention relates to the use of the whole cell catalytic system in the conversion of a hydantoin of formula [9]

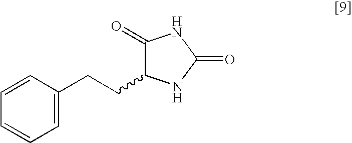

into an L-α-amino acid of formula [10]

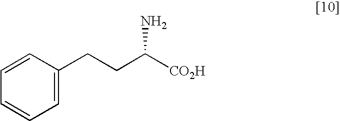

The compounds of formula [5], [6], [7] and [8] are novel compounds and are also part of the present invention.

In the present context “enantiomerically-enriched” means that one enantiomer or set of diastereomers preponderates over the complementary enantiomer or set of diastereomers.

In the present context “amino acid” means a compound in which at least one amino group and at least one carboxyl group are bound to the same carbon atom (α-C-atom)

### DESCRIPTION OF THE FIGURES

FIG. 1. A physical and functional map representing the basic construction of the operon (Hyu1 Operon). hyuH, hyuC and hyuA represent the genes coding for L-hydantoinase from Arthrobacter aurescens, L-carbamoylase from Bacillus stearothermophilus and hydantoin racemase from Agrobacterium radiobacter (HyuA), respectively. rbs means ribosomal binding site. NdeI and KpnI indicate restriction sites.

FIG. 2. A physical and functional map representing the basic construction of the expression vector pKECaro_hyu1. hyuH, hyuC and hyuA have the meanings described above. Km(R) is the kanamycin resistance gene. ori327 is the origin of replication from plasmid pBR327. aroH-P is the promoter of tryptophan-sensitive 3-deoxy-D-arabinoheptulosonic acid-7-phosphate synthetase. ClaI, SpeI, HpaI, NdeI, ApaI, EcoRI, KpnI, SalI, MluI, HindIII and Xho1 indicate restriction sites.

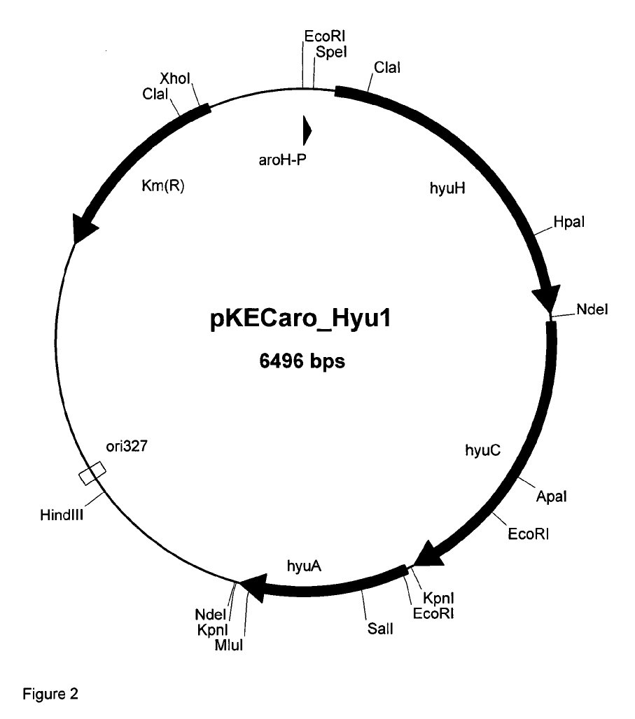

### MATERIALS AND METHODS

Unless indicated otherwise, all molecular techniques employed were essentially performed according to Maniatits et al. (J. Sambrook, E. F. Fritsch, T. Maniatis. Molecular Cloning 2^{nd} edition. CSH Press).

Protocol for Transformation of pKECaroP-hyu1 Construct into E. Coli RV308
Thaw E. coli RV308 aliquots (200 μl, supercompetent) on ice
Add 15 μl LR reaction mix (see above)
Incubate 30 minutes on ice
Heatshock 1 minute 42° C.
Cool cells 2 minutes on ice
Add 1 ml LB medium (5 g/l NaCl, 5 g/l yeast extract, 10 g/l tryptone)
Incubate 1 hour 37° C.
Plate on LB agar plates supplemented with kanamycine (5 g/l NaCl, 5 g/l yeast extract, 10 g/l tryptone, 15 g/l agar, 50 mg/l kanamycine)
Incubate 24 hours 28° C.
Isolate single colonies

### Protocol for Expression of Hyu Genes in E. Coli RV308

Single clones from the transformation (see above) were used to inoculate 5 ml of 2×TY media (10 g/l yeast extract, 16 g/l tryptone, 5 g/l NaCl) supplemented with 0.05 g/l kanamycine and 1 mM MnCl₂ or CoCl₂, respectively. The culture was incubated at 28° C. and 150 rpm for 24 hours and then used for inoculation of 100 ml 2×TY media supplemented with 0.05 g/l kanamycine and 1 mM MnCl₂ or CoCl₂, respectively. The cultures were again incubated for 24-28 hours under conditions previously mentioned and subsequently harvested by centrifugation (20 minutes, 5000 rpm, 4° C.). The cell pellet was resuspended in 5 ml Tris-HCl (100 mM, pH 7), centrifuged again (20 minutes, 5000 rpm, 4° C.) and the cells were frozen at −20° C.

### Examples

### Example 1

### Construction of a Clone for Co-Expression of L-Hydantoinase, L-Carbamoylase and Hydantoin Racemase in E. Coli RV308

The aim was to obtain active coexpression of the L-hydantoinase from Arthrobacter aurescens (HyuH), the L-carbamoylase from Bacillus stearothermophilus (HyuC) and the hydantoin racemase from Agrobacterium radiobacter (HyuA) in the host E. coli RV308 resulting in a production strain for the production of L-amino acids.

The sequences of the 3 enzymes are known from the following literature sources:
L-hydantoinase (represented below as SEQ ID NO: 2) from Abendrodt et al. Biochemistry 41 (27), 8589-8597 (2002)
L-carbamoylase (represented below as SEQ ID NO: 3) from Battise et al. Appl. Environ. Microbiol. 63(2), 763-766 (1997
Hydantoin racemase (represented below as SEQ ID NO: 4) from patent EP 1506294B1,

An operon (represented by SEQ ID NO: 1) was synthetically prepared wherein the three genes of the hydantoin pathway (hyuH, hyuC, hyuA) are separated from each other by spacers containing a ribosomal binding site rbs (Shine-Delgarno Sequence) and a restriction site for further subcloning.

The order of the subsequences in the construct is as follows:
Restriction site 1 (SEQ ID NO: 5);
Sequence coding for L-hydantoinase (SEQ ID NO: 2);
Spacer (SEQ ID NO: 6);
Sequence coding for L-carbamoylase (SEQ ID NO: 3);
Spacer (SEQ ID NO: 6);
Sequence coding for hydantoin racemase (represented below as SEQ ID NO 4) and
Rbs (SEQ ID NO: 7).

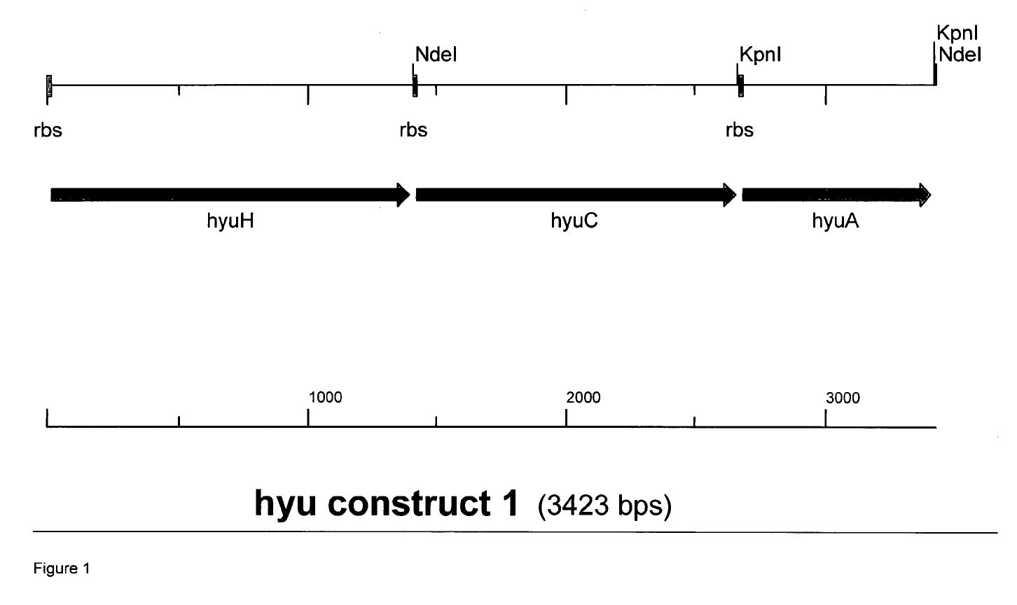

A map representing the basic construction of the operon (Hyu1 Operon) is illustrated in FIG. 1.

The DNA sequences of the enzyme-encoding regions were optimized for expression in E. coli RV308.

The Hyu1 operon was subsequently cloned into an expression vector. The expression vector pKECaro_hyu1 is derived from plasmid pKECtrp (described in WO 0066751) by replacing the trp promoter ==> PenG acylase expression cassette by the aroH promoter ==> hyul operon.

The DNA was transformed into supercompetent E. coli RV308 cells (as described in Material and Methods) and single clones were isolated from the agar plate. The clones were grown in LB medium supplemented with kanamycin (5 g/l NaCl, 5 g/l yeast extract, 10 g/l tryptone, 50 mg/l kanamycin) and plasmid DNA was isolated using the Qiagen Miniprep Kit (following the standard procedure). The accuracy of the constructs was checked by restriction analysis.

### Example 2

### Preparation of S-2-amino-non-8-enoic acid Synthesis of 5-hept-6-enyl-imidazolidin-2,4-dione [3]

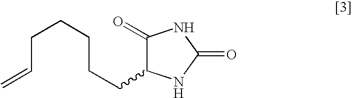

To a solution of 100,0 g ammonium carbonate and 29.4 g sodium cyanide in 950 ml of water and 300 ml of ethanol was added 63,0 g of 7-octenal. The two-phase system was heated to 60° C. and subsequently stirred overnight.

The mixture was allowed to cool to ambient temperature after which the precipitated white product was isolated by filtration, washed with water and subsequently dried in vacuo. The obtained crude product was purified by stirring in chloroform for 1 hour at ambient temperature. The hydantoin was isolated by filtration, washed with chloroform and dried in vacuo to yield 39.0 g of white solid. ¹H NMR (300 MHz, DMSO-d6) δ ppm): 10.62 (1H, NH), 7.92 (1H, NH), 5.81 (1H, m), 4.99 (2×1H, md, J=18.0 Hz, 9.0 Hz), 3.98 (1H, m), 2.01 (2H, m), 1.68 (1H, m), 1.53 (1H, m), 1.35 (6H, m). ¹³C-NMR (75 MHZ, DMSO-d6) δ ppm): 176.59, 157.99, 139.07, 115.06, 57.91, 33.38, 31.55, 28.85, 28.48, 28.41, 24.32, 22.41.

It should be noted that when the starting material 7-octenal is not 100% pure but also comprises amounts of octenal and 6-octenal, the hydantoin and amino acid derivates thereof are also produced.

### Synthesis of S-2-amino-non-8-enoic acid [4]

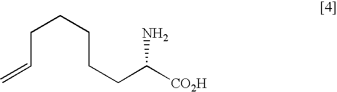

1.9 g of wet cells obtained according to the above Protocol for expression of Hyu genes in E. coli RV308 was suspended in 40 g Tris.HCl buffer (pH 8.5). Subsequently, 450 μl of a 100 mmol/l MnCl₂ solution and 1.0 g of 5-hept-6-enyl-imidazolidin-2,4-dione were added and the mixture was incubated overnight at 37° C. The mixture was cooled to 20° C. after which the pH was raised to 13 by the addition of 5 mol/l aqueous KOH. The cells were removed by centrifugation after which the supernatant was acidified to pH 6 by the addition of 4 N aqueous HCl. The precipitated product was isolated by filtration and subsequently washed with water and methanol to yield 0.8 g of off-white product (85% chemical yield), ee>99.5% (HPLC). ¹H NMR (300 MHz, D₂O) δ ppm): 5.56 (1H, tdd, J=18.0 Hz, 9.0 Hz), 4.82 (1H, md, J=18.0 Hz), 4.65 (1H, md, J=9.0 Hz), 3.77 (1H, m), 1.71 (2H, m), 1.62 (2H, m), 1.05 (6H, m).

### Example 3

### Preparation of S-phenyl alanine

0.28 g of wet cells was suspended in 3 ml of Tris.HCl buffer (pH 8.5). Subsequently, 30 μl of a 100 mM MnCl₂ solution and 57.5 mg g of 5-benzyl-imidazolidin-2,4-dione were added and the mixture was incubated overnight at 37° C. The mixture was cooled to 20° C. after which the sample was analyzed by chiral HPLC. The S-phenyl alanine thus prepared had an ee>99.8%.

### Example 4

### Preparation of (S)-2-amino-3-(2-oxocyclopentyl)propanoic acid

### 1. Synthesis of [5a]

L-Serine hydantoin(5-hydroxymethylimidazolidin-2,4-dione, 41.4 g, 318 mmol) was suspended in dry MeCN (330 mL) and the solution was cooled to 10° C. Pyridine (88.3 g, 1.11 mol) was then added and a homogeneous solution formed. Acetyl chloride (87.4 g, 1.11 mol) was added via addition funnel drop wise during ca. 1 h, so that the reaction temperature was maintained at 10° C. After the completion of the addition, the reaction was allowed to warm up to 20° C. (within ca. 30 min) while a precipitate formed. Slowly the reaction mixture became homogeneous again. After approx. 16 h, the reaction was complete (as determined by ¹H NMR in CDCl₃). It was then concentrated on the rotavapor at 40° C. to a final weight of ca. 240 g. Some solids formed. MTBE (500 mL) was then added and more solid precipitated. After stirring for 10 min, the precipitate (pyridinium chloride) was collected on a funnel via suction. The cake was washed with additional MTBE (100 mL) and then discarded. The product, tris-acetyl 5-hydroxymethylimidazolidin-2,4-dione, resided in the mother liquor. This was concentrated on a rotavapor at 40° C. to a thick oil and used as such in the next step. Weight 73.0 g, ca. 95% purity, approx. 85% yield. ¹H NMR: (CDCl₃, 300 MHz): δ 4.83 (dd, 1H), 4.73 (t, 1H), 4.44 (dd, 1H), 2.61 (s, 3H), 2.60 (s, 3H), 2.03 (s, 3H).

Tris-acetyl 5-hydroxymethylimidazolidin-2,4-dione obtained above (6.06 g, 95% pure, 22.5 mmol) was dissolved in THF (25 mL) and added drop wise via an addition funnel into a solution of triethylamine (3.57 g, 35.3 mmol) and 1-pyrrolidino-1-cyclopentene (4.85 g, 35.3 mmol) in THF (25 mL) at 20° C. over a period of approx. 45 min. The homogeneous, yellow-orange reaction was stirred further for 16 h at 20° C. The conversion was complete as determined by ¹H NMR in DMSO-d₆. Then, water (8 mL) was added followed by conc. aq. HCl (3 mL). After stirring for 30 min, the solvent was removed on the rotavapor at 40° C. Additional water (15 mL) was added and the product was extracted into EtOAc (2×25 mL) and the combined organic extracts were dried over anhydrous Na₂SO₄. After filtration and concentration of the solution on the rotavapor at 60° C., the product, N₄-acetyl-[5a] was purified by silica gel flash chromatography using an EtOAc:petroleum benzene gradient. Weight 4.35 g, 81% yield. The product N₄-acetyl-[5a] is a ca. 1:1 mixture of diastereomers. ¹H NMR: (CDCl₃, 300 MHz): δ 8.49 (br s, 1H), 4.82 (dd, 0.5H), 4.63 (dd, 0.5H), 2.57 (s, 3H), 2.46-2.00 (m, 7H), 1.88-1.75 (m, 1H), 1.67-1.53 (m, 1H).

N₄-acetyl-[5a] (0.500 g, 2.10 mmol) was dissolved in 6M aq. HCl and was stirred at 80° C. for 16 h. The conversion was followed by TLC on silica-coated plates (eluent is 7:3 EtOAc:petroleum benzene). The reaction mixture was then cooled to 20° C. and extracted with ethyl ether (10 mL). The organic layer was discarded. The aqueous phase was concentrated on the rotavapor at 70° C. to give 5-((2-oxocyclopentyl)methyl)imidazolidine-2,4-dione [5a] (mixture of diastereomers) as a brownish solid. Weight 0.360 g, 87% yield. ¹H NMR: (DMSO-d₆, 300 MHz): δ 10.63 (br s, 1H), 7.99 (br s, 0.73H, major diastereomer), 7.90 (br s, 0.27H, minor diastereomer), 4.19 (td, 0.27H, minor diastereomer), 4.06 (ddd, 0.73H, major diastereomer), 2.30-2.02 (m, 4H), 1.98-1.80, 2H), 1.77-1.62 (m, 1H), 1.60-1.40 (m, 2H).

### 2. Synthesis of (S)-2-amino-3-(2-oxocyclopentyl)propanoic acid [6a]

5-((2-oxocyclopentyl)methyl)imidazolidine-2,4-dione [5a] (0.20 g, 1.0 mmol) was suspended in TRIS-HCl buffer (400 mM, 5 mL) at pH 8.5. Then, 0.8 g of wet cell slurry obtained according to ‘Protocol for expression of Hyu genes in Escherichia coli RV308’ in ‘Materials and methods’ was added, followed by 50 μL of a 100 mmol/L MnCl₂ solution and the mixture was incubated overnight at 37° C. The mixture was then centrifuged at room temperature and the supernatant was filtered through a 0.45 μ filter. The product aminoacid [6a] has the S configuration at C2 with >99% ee (the other chiral center C4 is scrambled).

### Example 5

### Preparation of (S)-2-amino-3-(2-oxocyclohexyl)propanoic acid

### 1. Synthesis of [7a]

Compound [7a] was prepared similarly, with similar yield, as described above for compound [5a] however using 1-pyrrolidino-1-cyclohexene rather than 1-pyrrolidino-1-cyclopentene.

### 2. Synthesis of (S)-2-amino-3-(2-oxocyclohexyl)propanoic acid [8a]

Compound [8a] was prepared from [7a] similarly, with similar yield, as described above for the synthesis of compound [6a] from compound [5a].

1. Whole cell catalytic system for preparation of enantiomerically enriched α-amino acid from a corresponding hydantoin wherein hydantoinase, L-carbamoylase, and hydantoin racemase are coexpressed in a recombinant microorganism; wherein genes coding for these three enzymes are located on a single replicon; characterized in that the hydantoin racemase is derived from an Agrobacterium species.

2. Whole cell catalytic system according to claim 1, wherein the recombinant microorganism is E. coli.

3. Whole cell catalytic system according to claim 1, wherein the carbamoylase and the hydantoinase are derived from a species other than an Agrobacterium species.

4. Whole cell catalytic system according to claim 1, characterized by the coexpression of hydantoinase represented by SEQ ID NO: 2 or by a protein at least 90% identical to SEQ ID NO: 2, carbamoylase represented by SEQ ID NO: 3 or by a protein at least 90% identical to SEQ ID NO: 3, and hydantoin racemase represented by SEQ ID NO: 4 or by a protein having a homology of at least 90% identical to SEQ ID NO: 4.

5. Whole cell catalytic system according to claim 3, wherein the recombinant microorganism is E. coli.

6. Whole cell catalytic system according to claim 4, wherein the recombinant microorganism is E. coli.

7. (canceled)

8. DNA construct comprising nucleotide sequences encoding hydantoinase, L-carbamoylase, and hydantoin racemase operationally linked to a single promoter; characterized in that the hydantoin racemase is derived from an Agrobacterium species.

9. Expression vector containing a DNA construct according to claim 8.

10. DNA construct according to claim 8, wherein the hydantoinase and the L-carbamoylase are derived from a species other than an Agrobacterium species.

11. DNA construct according to claim 8, wherein the nucleotide sequences encode hydantoinase represented by SEQ ID NO: 2, carbamoylase represented by SEQ ID NO: 3, and hydantoin racemase represented by SEQ ID NO: 4.

12. Expression vector containing a DNA construct according to claim 10.

13. (canceled)

14. Method for preparation of enantiomerically enriched α-amino acid comprising conversion of a hydantoin of the general formula [1] 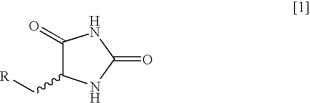
into an L-α-amino acid of general formula [2] 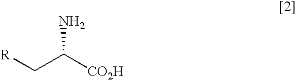
wherein R is a substituent with at least 3 carbon atoms, optionally containing further one or more heteroatoms, one or more double bonds, and or one or more cyclic structures using a whole cell catalytic system according to claim 1.

15. Method according to claim 14, wherein R is CH₂═CHCH₂CH₂CH₂CH₂—, PhCH₂—, 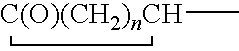
wherein n is 3 or 4, or 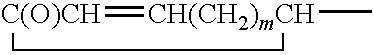
wherein m is 1 or 2.

16-17. (canceled)

18. A compound of formula [1] 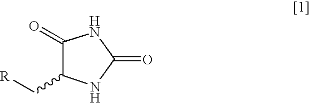
wherein R is CH₂═CHCH₂CH₂CH₂CH₂— or 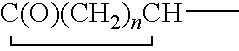
wherein n is 3 or 4 or 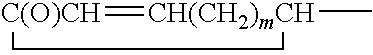
wherein m is 1 or 2.

19. A compound of formula [2] 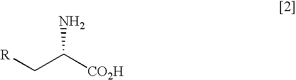
wherein R is CH₂═CHCH₂CH₂CH₂CH₂— or 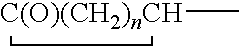
wherein n is 3 or 4 or 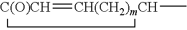
wherein m is 1 or 2.

20-23. (canceled)

24. Method according to claim 14, wherein R is 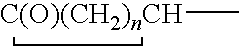
wherein n is 3 or 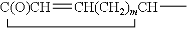
wherein m is 1; characterized in that the L-α-amino acid of formula [2] is further hydrogenated to give (2S,3aS,6aS)-octahydrocyclopenta[b]pyrrole-2-carboxylic acid.

25. Method according to claim 14, wherein R is 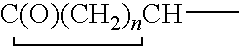
wherein n is 4 or 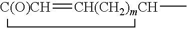
wherein m is 2; characterized in that the L-α-amino acid of formula [2] is further hydrogenated to give (2S,3aS,6aS)-decahydrocyclohexa[b]pyrrole-2-carboxylic acid.

26. Method for preparation of enantiomerically enriched α-amino acid comprising conversion of a hydantoin of the general formula [1] 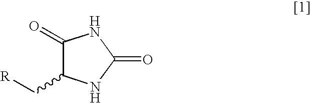
into an L-α-amino acid of general formula [2] 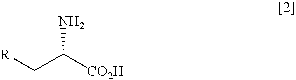
wherein R is a substituent with at least 3 carbon atoms, optionally containing further one or more heteroatoms, one or more double bonds, and or one or more cyclic structures using a whole cell catalytic system according to claim 3.

27. Method according to claim 26, wherein R is CH₂═CHCH₂CH₂CH₂CH₂—, PhCH₂—, 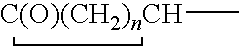
wherein n is 3 or 4, or 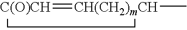
wherein m is 1 or 2.

28. Method according to claim 26, wherein R is 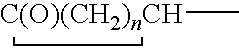
wherein n is 3 or 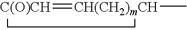
wherein m is 1; characterized in that the L-α-amino acid of formula [2] is further hydrogenated to give (2S,3aS,6aS)-octahydrocyclopenta[b]pyrrole-2-carboxylic acid.

29. Method according to claim 26, wherein R is 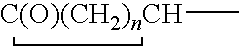
wherein n is 4 or 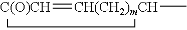
wherein m is 2; characterized in that the L-α-amino acid of formula [2] is further hydrogenated to give (2S,3aS,6aS)-decahydrocyclohexa[b]pyrrole-2-carboxylic acid.

30. Whole cell catalytic system for preparation of enantiomerically enriched α-amino acid from a corresponding hydantoin wherein hydantoinase, L-carbamoylase, and hydantoin racemase are coexpressed in a recombinant microorganism; wherein genes coding for these three enzymes are located on a single replicon; wherein the hydantoin racemase is derived from an Agrobacterium species; wherein nucleotide sequences encoding hydantoinase, L-carbamoylase, and hydantoin racemase are operationally linked to a single promoter; and wherein said genes are contained in an expression vector.

31. Method for preparation of enantiomerically enriched α-amino acid comprising conversion of a hydantoin of the general formula [1] 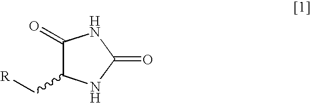
into an L-α-amino acid of general formula [2] 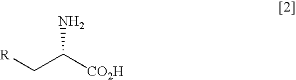
wherein R is a substituent with at least 3 carbon atoms, optionally containing further one or more heteroatoms, one or more double bonds, and or one or more cyclic structures using a whole cell catalytic system according to claim 30.


SEQUENCE LISTING (7 sequences)

SEQ ID NO: 1 - DNA, 3423 bp, Artificial Sequence
  synthetic polynucleotide
aggaggaattaaccatgtttgatgtaatcgtgaaaaattgccgtctggtatcttccgacggtattaccgaagcagatatcctggtcaaggatggcaaagtggcggctatctccgcagacaccagcgacgtggaagcgagccgtaccatcgatgctggtggcaagttcgtaatgccgggtgtagtggacgaacacgtgcacattatcgacatggacctgaaaaaccgctatggtcgcttcgaactggattccgaatctgctgcagttggtggcatcaccaccatcatcgaaatgccaattaccttccctccgaccaccaccctggatgcctttctggaaaagaaaaagcaggcaggtcagcgcctgaaagtggatttcgcactgtacggcggtggtgtgccgggtaacctgccggagatccgtaagatgcacgatgcaggtgctgtcggcttcaaaagcatgatggcggcttccgtcccgggtatgttcgatgcggtttccgacggtgaactgttcgaaatctttcaggaaattgcggcctgtggtagcgttatcgtggttcacgctgaaaacgaaacgatcattcaggctctgcagaaacaaattaaggctgctggcggcaaggacatggctgcttacgaggcgagccagccagttttccaagaaaatgaggcgatccagcgcgctctgctgctgcagaaagaagcaggttgccgcctgatcgtgctgcacgttagcaacccggacggtgtggagctgatccatcaagcacagtccgagggtcaggacgttcactgcgagtctggcccgcaatacctgaacatcactaccgatgatgcagaacgtatcggtccatatatgaaggtagccccgccagtccgttccgccgaaatgaacattcgtctgtgggaacaactggaaaacggcctgatcgacaccctgggctctgatcacggcggccacccagttgaggacaaagaaccgggctggaaggatgtttggaaggcgggtaacggcgccctgggcctggaaactagcctgccgatgatgctgactaacggtgttaacaaaggccgtctgtccctggaacgtctggttgaggttatgtgcgaaaaaccagctaagctgtttggcatctacccgcagaaaggcaccctgcaggtcggttctgacgcggatctgctgatcctggatctggatattgacacgaaggtcgatgcttctcagttccgttctctgcacaagtattctccgttcgacggtatgccggtaaccggcgcaccggtactgacgatggtacgtggtacggtggtagccgaaaaaggtgaagttctggttgagcagggcttcggccagttcgttactcgtcgtaactatgaagcaagcaaataaatctaagttatcatatgaggaggaattaaccatgattcaaggtgaacgtctgtggcagcgtctgatggaactgggtgaagtcggtaaacagccgagcggcggcgttactcgcctgtccttcactgcggaagaacgccgtgccaaagacctggttgccagctatatgcgcgaagctggtctgtttgtatacgaagacgcagcaggcaacctgatcggccgtaaagaaggcaccaacccggatgcaaccgtggttctggttggttctcatctggactctgtctacaacggcggttgtttcgatggtccgctgggtgttctggctggcgttgaggttgtgcagactatgaacgaacacggtgtggtcacgcaccacccgattgaagttgttgcgttcaccgatgaagaaggtgcgcgcttccgttttggcatgattggcagccgcgcaatggctggcaccctgccgcctgaagctctggaatgtcgtgacgcagaaggtatcagcctggcggaggctatgaagcaggcgggtctggacccagatcgtctgccgcaagcagcacgcaaaccaggcaccgtgaaagcgtacgttgaactgcacatcgaacagggtcgtgtactggaagaggctggtctgccagtaggtatcgtgaccggcattgcgggtctgatctgggttaaatttactattgcgggccctgccgagcacgccggtgcaacgccgatgagcctgcgccgcgacccgatggccgcagctgcccagatcattatcgtgatcgaggaagaagctcgtcgtaccggcaccaccgtaggcaccgttggtcagctgcacgtttacccgggtggtattaacgttatcccggaacgtgtagaattcgtgctggatctgcgtgatctgaaggccgaagttcgtgaccaagtgtggaaagcgatcgcagttcgtgccgaaaccattgctaaagaacgcaacgtacgcctgactaccgaacgtctgcaggaaatggcaccggttctgtgttccgaagttgtgaaacaagcggcagagcgtgcatgcaaacagctgggctacccgccgttttggctgccatctggcgcggcccacgacggcgtgcagctggcgccgatctgcccgatcggcatgatctttgtccgtagccaagatggcgtttctcactctccggccgaatggtccaccaaagaagactgcgctgttggtgcggaggttctgtatcacactgtttggcagctggctcagggtgaataaatctaagttatggtaccaggaggaattaaccatgcatatccgtctgattaacccgaattctactgcctctatgaccgcacaggcgctggatagcgcactgcgcgtaaaacaggcacataccactatctctgcaactaacccgctggacacgccggtgtctatcgaaggcggtgcagacgaagcactggctgtaccgggcatgctggaagaaattcgtaaaggcgagcgtctgggtgtcgacgcttacgtgatcgcatgcttcgacgacccgggcctgcacgctgcccgtgaagttgcgcgcggtccggtaatcggtatctgccaggccggcattcaggtagctatgaccatcagccgccgttttagcatcatcacgaccctgccgcgttccatcccgatcatcgaagacctggtcgataactatggcgcgcagcgtcattgccgtcgtgtccgtgccattaacctgccggttctgggtctggaagaagacccgcatgctgctgaagcaatgctgattcgcgagatcgaggcggcgaaaaaggaagacgccgcggaggcgatcattctgggttgcgccggcatgtctgctctgtgcgatcgtctgcgcgaagcgactggtgttccggtgatcgacggtgttaccgcggcagtaaaactggcagaagccctggtgggcgctggttacagcactagcaaagtgaacgcgtacgattacccgcgtatcaaagaaaccaaactggtggcctaatagcaagtttaggtacccatatg

SEQ ID NO: 2 - protein, 458 aa, Artificial Sequence
  synthetic polypeptide
MFDVIVKNCRLVSSDGITEADILVKDGKVAAISADTSDVEASRTIDAGGKFVMPGVVDEHVHIIDMDLKNRYGRFELDSESAAVGGITTIIEMPITFPPTTTLDAFLEKKKQAGQRLKVDFALYGGGVPGNLPEIRKMHDAGAVGFKSMMAASVPGMFDAVSDGELFEIFQEIAACGSVIVVHAENETIIQALQKQIKAAGGKDMAAYEASQPVFQENEAIQRALLLQKEAGCRLIVLHVSNPDGVELIHQAQSEGQDVHCESGPQYLNITTDDAERIGPYMKVAPPVRSAEMNIRLWEQLENGLIDTLGSDHGGHPVEDKEPGWKDVWKAGNGALGLETSLPMMLTNGVNKGRLSLERLVEVMCEKPAKLFGIYPQKGTLQVGSDADLLILDLDIDTKVDASQFRSLHKYSPFDGMPVTGAPVLTMVRGTVVAEKGEVLVEQGFGQFVTRRNYEASK

SEQ ID NO: 3 - protein, 409 aa, Artificial Sequence
  synthetic polypeptide
MIQGERLWQRLMELGEVGKQPSGGVTRLSFTAEERRAKDLVASYMREAGLFVYEDAAGNLIGRKEGTNPDATVVLVGSHLDSVYNGGCFDGPLGVLAGVEVVQTMNEHGVVTHHPIEVVAFTDEEGARFRFGMIGSRAMAGTLPPEALECRDAEGISLAEAMKQAGLDPDRLPQAARKPGTVKAYVELHIEQGRVLEEAGLPVGIVTGIAGLIWVKFTIAGPAEHAGATPMSLRRDPMAAAAQIIIVIEEEARRTGTTVGTVGQLHVYPGGINVIPERVEFVLDLRDLKAEVRDQVWKAIAVRAETIAKERNVRLTTERLQEMAPVLCSEVVKQAAERACKQLGYPPFWLPSGAAHDGVQLAPICPIGMIFVRSQDGVSHSPAEWSTKEDCAVGAEVLYHTVWQLAQGE

SEQ ID NO: 4 - protein, 238 aa, Artificial Sequence
  synthetic polypeptide
MHIRLINPNSTASMTAQALDSALRVKQAHTTISATNPLDTPVSIEGGADEALAVPGMLEEIRKGERLGVDAYVIACFDDPGLHAAREVARGPVIGICQAGIQVAMTISRRFSIITTLPRSIPIIEDLVDNYGAQRHCRRVRAINLPVLGLEEDPHAAEAMLIREIEAAKKEDAAEAIILGCAGMSALCDRLREATGVPVIDGVTAAVKLAEALVGAGYSTSKVNAYDYPRIKETKLVA

SEQ ID NO: 5 - DNA, 14 bp, Artificial Sequence
  synthetic primer
aggaggaattaacc

SEQ ID NO: 6 - DNA, 31 bp, Artificial Sequence
  synthetic primer
atctaagttatcatatgaggaggaattaacc

SEQ ID NO: 7 - DNA, 23 bp, Artificial Sequence
  synthetic primer
tagcaagtttaggtacccatatg